(19) United States
(12) Patent Application Publication
(10) Pub. No.: US 2015/0094729 A1
(43) Pub. Date: Apr. 2, 2015

(54) ACETABULAR CUP INSERTER

(71) Applicant: DEPUY SYNTHES PRODUCTS, LLC, Raynham, MA (US)

(72) Inventor: MATTHEW D. SCHMIT, Fort Wayne, IN (US)

(21) Appl. No.: 14/040,824

(22) Filed: Sep. 30, 2013

(51) Int. Cl. A61F 2/46

(52) U.S. Cl. CPC A61F 2/4609
USPC 606/91

(57) ABSTRACT

An instrument for positioning a hollow cup component of an orthopaedic joint prosthesis. The instrument includes an insertion end section, a handle end section opposite the insertion end section, and a shaft coupling the insertion end section to the handle end section. The insertion end section includes a cam wheel and a plurality of expanding pieces coupled to the cam wheel such that when the cam wheel is rotated in one direction, the expanding pieces expand out in a radial direction and frictionally engage an inner surface of the hollow cup component.

### CROSS-REFERENCE INFORMATION

### Technical Field of the Invention

The present invention relates generally to the field of orthopedics, and, more particularly, to an instrument for inserting a cup into an acetabulum.

### Background

Certain orthopaedic joint prostheses include a hollow cup with an inner surface which defines a generally hemispherical hollow region, and another component which has a spherical part which can be received in the hollow region for articulation relative to the cup component. Such joint prostheses can include hip joint prostheses and shoulder joint prostheses. The exterior of the cup will contact the prepared surface of the patient's bone in which the component is to be implanted. The interior of the cup will present a smooth bearing surface to the spherical part of the other component of the joint prosthesis. The bearing surface can be provided by a single piece cup component. Alternatively, the cup component can comprise a shell part which contacts the prepared surface of the patient's bone, and a bearing part which provides the bearing surface, and which fits into the shell part. The bearing part can be made from a material which is different from the material of the shell part: for example the bearing part can be made from a polymeric material (such as polyethylene) and the shell part (and the spherical part of the other component) can be made from a metal (such as a cobalt-chromium based alloy, or a stainless steel, or a titanium based alloy).

It is important that the components of an orthopaedic joint prosthesis are positioned accurately in a patient's bone. Both location and alignment are important. Accurate positioning of a component requires that the component be engaged by an appropriate instrument, allowing considerable force to be applied to the component if and as necessary. However, it can be important not to contact the external surface or the internal surface or both of the component with the instrument, especially the internal surface when it has been provided with a smooth polished bearing surface. Scratching or otherwise damaging that surface can impair the bearing properties of the prosthesis.

In some prior art instruments, the insertion tool includes a flange that interacts with a groove cut into an inner surface of the cup. Other prior art instruments utilize a plurality of jaw members which extend radially from a central drive shaft. The jaw members can be made to slide radially inwardly so that they engage the outside wall of a cup component. Each of the jaw members has a pin at one end which is received in a spiral track on a drive plate. The jaw members are made to slide radially by rotating the drive plate. The jaw members engage a corresponding recess in the cup.

In each of these prior art examples, the insertion tool works by interacting with special features on the cup. This limits the instrument to only be able to be used with certain cups. Therefore, there is a need for an insertion instrument that can be used on a cup

### SUMMARY OF THE INVENTION

According to one embodiment, an instrument for positioning a hollow cup component of an orthopaedic joint prosthesis is provided. The instrument includes an insertion end section, a handle end section opposite the insertion end section, and a shaft coupling the insertion end section to the handle end section. The insertion end section includes a cam wheel and a plurality of expanding pieces coupled to the cam wheel such that when the cam wheel is rotated in one direction, the expanding pieces expand out in a radial direction and frictionally engage an inner surface of the hollow cup component.

According to yet another embodiment, a system including a hollow cup having a concave inner surface and an instrument for positioning the hollow cup component is provided. The instrument includes an insertion end section and a handle end section opposite the insertion end section. A shaft couples the insertion end section to the handle end section, wherein the insertion end section includes a cam wheel and a plurality of expanding pieces coupled and adjacent to the cam wheel such that when the cam wheel is rotated in one direction, the expanding pieces expand out in a radial direction and frictionally engage the concave inner surface of the hollow cup.

According to yet another embodiment of the present invention, method for inserting a hollow cup into an acetabulum is provided. An instrument is used and the instrument has an insertion end section, a handle end section opposite the insertion end section, and a shaft coupling the insertion end section to the handle end section. The insertion end section includes a cam wheel and a plurality of irregular shaped expanding pieces coupled to the cam wheel. The insertion end section is inserted into the inner surface of the hollow cup. A knob extending from a portion of the shaft on the instrument is rotated in a first direction. Rotating the knob causes the plurality of irregular shaped expanding pieces to expand and frictionally engage the inner surface of the cup. The instrument is used to insert the cup into a patient's acetabulum. The knob is rotated in a direction opposite the first direction, causing the irregular shaped expanding pieces to release the inner surface of the cup.

### BRIEF DESCRIPTION OF THE DRAWINGS

FIG. 1 is a cross-sectional view of a cup inserting instrument according to one embodiment of the present invention.

FIG. 2 is an exploded view of the instrument of FIG. 1.

FIG. 3 is a cross-sectional view of an end of the instrument of FIG. 1 with a cup.

FIG. 4 is a cross-sectional view of the instrument of FIG. 1.

FIG. 5 is a flow chart illustrating a method of using the instrument according to one embodiment of the present invention.

### DETAILED DESCRIPTION

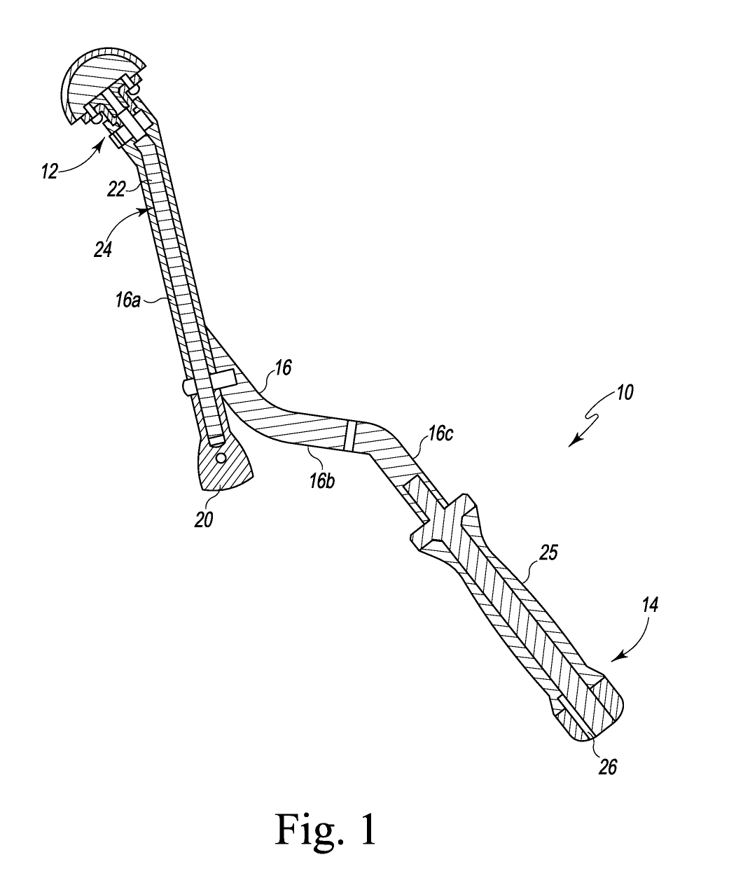

Like reference numerals refer to like parts throughout the following description and the accompanying drawings. Referring now to FIG. 1, an insertion instrument 10 is shown. As shown, the instrument 10 includes an insertion end section 12 and a handle end section 14 opposite the insertion end section 12. A shaft 16 couples the insertion end section 12 to the handle end section 14. In the illustrated embodiment, the shaft 16 includes three portions: a first portion 16a coupled to the insertion end section 12; a second portion 16b coupled to the first portion 16a, and a third portion 16c, coupled to the second portion 16b and the handle end section 14. As shown, the first portion 16a extends at an angle from the insertion end. The second portion is bent and extends away from the first portion. The third portion 16c is an angle to the second portion 16b and is in-line with the insertion end section 12. The three portions 16a, 16b, 16c may be one piece or they may be modular. In other embodiments, the shaft 16 may be a single portion in a straight line. The design of the shaft will depend upon the surgical procedure and the wound site.

As shown in FIG. 1, a knob 20 extends from the first portion 16a. The knob 20 is coupled to an internal rod 22 that extends through a cavity 24 in the first portion 16a of the shaft 16. Also as shown in FIG. 1, the handle end section 14 includes a handle 25 adapted to be gripped by a user during the use of the tool. The handle 25 may be ergonomically shaped for ease of handling. At the very end of the handle end section 14 is an impaction surface 26. The impaction surface 26 is structured to be able to be struck by a hammer, mallet or other impaction tool during use and to transfer the force through the shaft to the insertion end section 12.

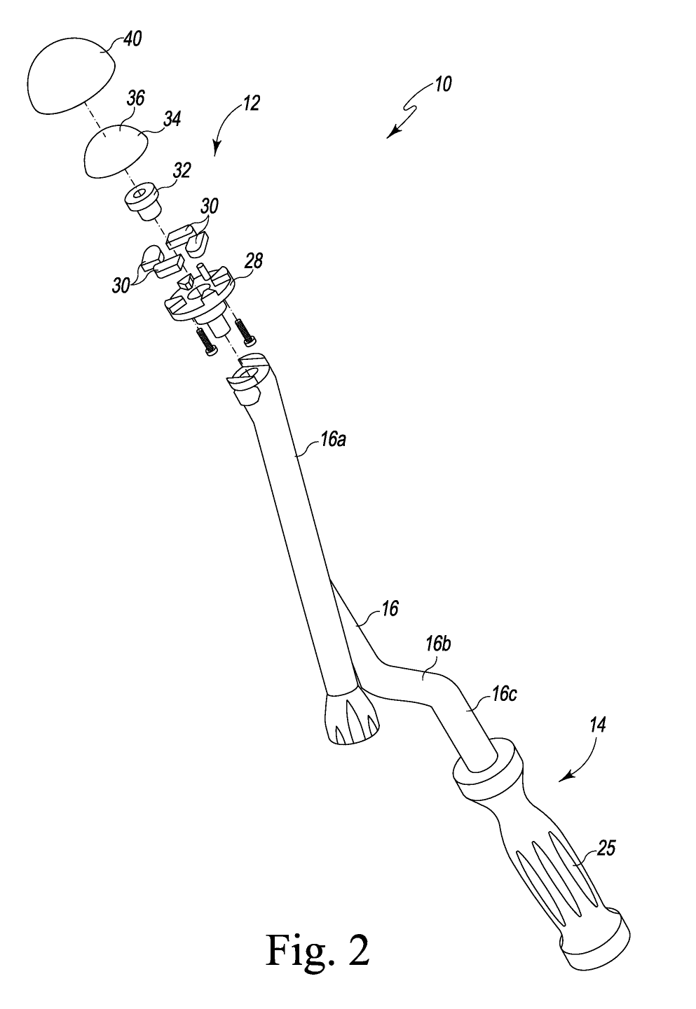

Turning now to FIG. 2, an exploded view of the instrument 10, especially the insertion end section 12 is shown. Coupled to the first portion 16a of the shaft 16 is a base plate 28. The base plate 28 provides support to the other components located at the insertion end section 12. The base plate 28 is coupled to a plurality of expanding pieces 30. The expanding pieces 30 have irregular shapes such that as they are rotated, the distance between the center of the rotation and the expanding piece 30 changes. Located in the middle of the expanding pieces is a cam wheel 32. The cam wheel 32 is coupled to the internal rod 22 (FIG. 1). When the knob 20 is rotated, the internal rod 22 rotates, rotating the cam wheel 32. The cam wheel 32 acts as a center of rotation for the expanding pieces 30. As the cam wheel 32 is rotated, the expanding pieces expand so that an edge expands beyond the edge of the base plate 28. In other words, as the cam wheel 32 rotates, the expanding pieces 30 expand inwardly and outwardly in a radial direction.

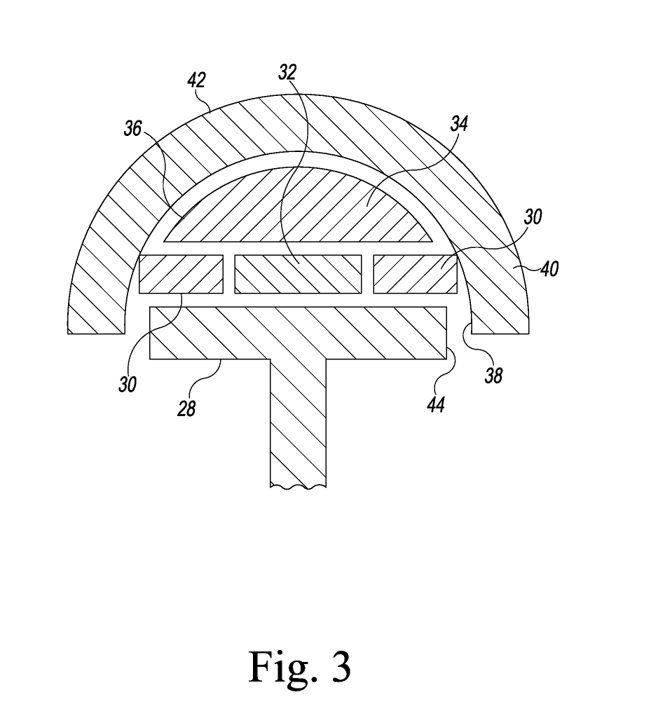

Also shown in FIG. 2 is an impactor cap 34. The impactor cap 34 has a partially spherical convex outer surface 36. The outer surface 36 is designed to engage an inner surface 38 (FIG. 3) of an acetabular cup 40.

FIG. 3 illustrates a perspective cut-away view of the insertion end section 12. As shown, the base plate 28 supports the cam wheel 32 and the expanding pieces 30. FIG. 3 illustrates an expanded state in which the cam wheel 32 has been rotated to cause the expanding pieces to extend beyond the base plate 28 and to engage the inner surface 38 of the acetabular cup 40.

In the embodiment illustrated in FIG. 3, the cup 40 is a monoblock cup. The outer surface 42 is made of a biocompatible metal, such as titanium. The outer surface 42 may also have a porous coating as is known in the art to promote bone in-growth. The inner surface 38 may be made of a polyethylene, such as antioxidant polyethylene. Other polyethylenes, such as ultra high molecular weight polyethylene (UHMWPE) may also be used. In this embodiment, the polyethylene inner surface 38 is molded into the metal outer surface 42.

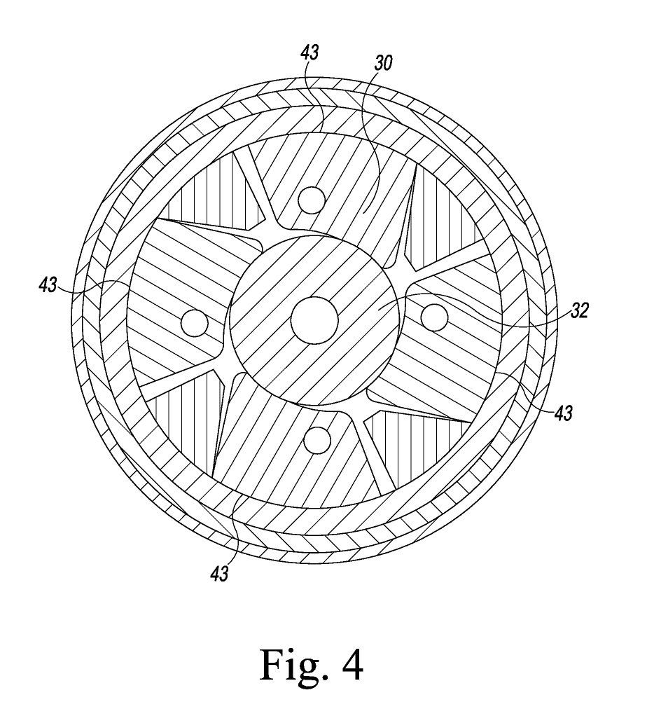

FIG. 4 shows the interaction between the cam wheel 32 and the expanding pieces 30 in more detail. As shown, the expanding pieces have edges 43. As the cam wheel 32 rotates, the edges 43 are engaged. Because the edges 43 of the expanding pieces 32 are not spherical, the expanding pieces move relative to the base plate 28. Specifically, the expanding pieces 30 are forced to move in and out relative to an edge 44 (FIG. 3) of the base plate 28.

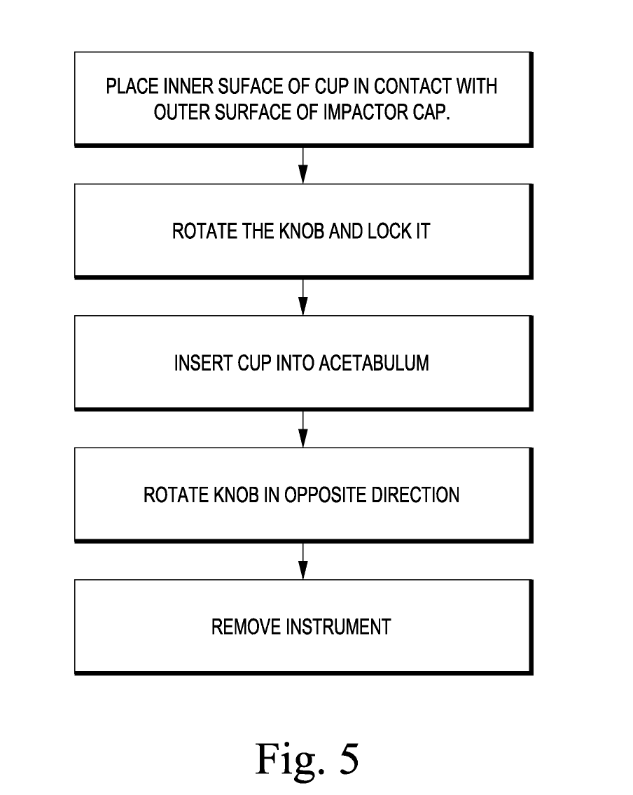

Turning now to FIG. 5, the opearation of the insertion instrument 10 will be described. Beginning at step s100, the inner surface 38 of the cup 40 is placed in contact with the outer surface 36 of the impactor cap 34. The user then rotates the knob 20 and locks it at step s102. Internally, the rotation of the knob 20 causes the internal rod 22 to rotate, which in turn causes the cam wheel 32 to rotate. As discussed above, as the cam wheel 32 rotates, expanding pieces 30 expand outward, entering into a friction fit with the inner surface 38 of the cup 40. At step 104, the user may then use the instrument 10 to insert the cup 40 into the prepared acetabulum. This step may involve using a hammer, mallet or other tool to strike the tool to firmly seat the shell in the acetabulum. Once the cup 40 is seated, the user then rotates the knob 20 in the opposite direction at step s106, causing the expanding pieces 30 to move back in toward the center and releases the friction fit between the expanding pieces 30 and the inner surface 38 of the cup 40. This step disengages the instrument 10 from the cup 40 and the instrument can be removed at step s108

Although the instrument of the present embodiment is used to describe inserting a cup, the instrument 10 may also be used to remove a cup. For example, if after the cup 40 is inserted into the acetabulum, the user is unhappy with the position, the user may insert the insertion end section 12 into the cup 40, turn the knob 20 in a direction to cause the expanding pieces 30 to engage the inner surface 38 of the cup 40, and then remove the instrument 10 and the cup 40 from the acetabulum. This is best if done prior to any impaction.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. An instrument for positioning a hollow cup component of an orthopaedic joint prosthesis, which comprises:
an insertion end section;
a handle end section opposite the insertion end section; and
a shaft coupling the insertion end section to the handle end section;
wherein the insertion end section includes a cam wheel and a plurality of expanding pieces coupled to the cam wheel such that when the cam wheel is rotated in one direction, the expanding pieces expand out in a radial direction and frictionally engage an inner surface of the hollow cup component.

2. The instrument according to claim 1, wherein the insertion end section further includes a base plate coupled to the cam wheel and expandable pieces.

3. The instrument according to claim 1, wherein the shaft includes a cavity and an internal rod extends at least partially through the internal cavity and is coupled to the cam wheel.

4. The instrument according to claim 3, wherein a knob extends from a portion of the shaft and the knob is coupled to the internal rod, such that as the knob is rotated, the internal rod rotates.

5. The instrument according to claim 1, wherein the shaft includes a plurality of portions, each of the portions at an angle with the adjacent portion.

6. The instrument according to claim 1, wherein the handle end portion includes an ergonomically shaped handle.

7. The instrument according to claim 6, wherein an end of the handle includes an impaction plate.

8. The instrument according to claim 1, wherein the insertion end section includes an impaction cap, the impaction cap being sized and shaped to engage an inner surface of the hollow cup.

9. A system including:
a hollow cup having a concave inner surface; and
an instrument for positioning the hollow cup component, the instrument including an insertion end section,
a handle end section opposite the insertion end section, and
a shaft coupling the insertion end section to the handle end section, wherein the insertion end section includes a cam wheel and a plurality of expanding pieces coupled and adjacent to the cam wheel such that when the cam wheel is rotated in one direction, the expanding pieces expand out in a radial direction and frictionally engage the concave inner surface of the hollow cup.

10. The system of claim 9, wherein the hollow cup is a monoblock cup having a metal outer surface and a polyethylene liner molded to the outer surface.

11. The system of claim 9, wherein the insertion end section further includes a base plate coupled to the cam wheel and the expanding pieces.

12. The system of claim 11, wherein the base plate includes an edge and each of the expanding pieces have edges, wherein when the cam wheel is rotated, the edges of each of the plurality of expanding pieces move relative to the edge of the base plate.

13. The system of claim 9, wherein the shaft includes a cavity and an internal rod extends at least partially through the internal cavity and is coupled to the cam wheel.

14. The system of claim 13, wherein the internal rod is coupled to a knob which extends from a portion of the shaft.

15. A method for inserting a hollow cup into an acetabulum, comprising:
using an instrument, the instrument having an insertion end section, a handle end section opposite the insertion end section, a shaft coupling the insertion end section to the handle end section, wherein the insertion end section includes a cam wheel and a plurality of irregular shaped expanding pieces coupled to the cam wheel;
inserting the insertion end section of the instrument into the inner surface of the hollow cup;
rotating a knob in a first direction extending from a portion of the shaft on the instrument, wherein rotating the knob causes the plurality of irregular shaped expanding pieces to expand and frictionally engage the inner surface of the cup;
using the instrument to insert the cup into a patient's acetabulum; and
rotating the knob in a direction opposite the first direction, causing the irregular shaped expanding pieces to release the inner surface of the cup.

16. The method of claim 15, wherein the shaft includes an internal rod coupled to the knob and the cam wheel, such that when the knob is rotated, the internal rod rotates, causing the cam wheel to rotate.

17. The method of claim 15, wherein each of the irregular shaped expanding pieces have edges and as the irregular shaped expanding pieces is rotated, the edges of each of the plurality of irregular shaped expanding pieces extend into frictional engagement with the inner surface of the cup.

18. The method of claim 15, wherein the insertion end section includes an impaction cap and the impaction cap engages a portion of the inner surface of the cup.

19. The method of claim 15, wherein the shaft includes a plurality of portions, each of the portions extending at an angle from the adjacent portion.

20. The method of claim 15, further comprising striking the handle end portion with a tool to impact the cup into the acetabulum.
